(19) United States
(12) Patent Application Publication
(10) Pub. No.: US 2025/0073485 A1
(43) Pub. Date: Mar. 6, 2025

(54) LED PHOTOTHERAPY DEVICE CONTROL SYSTEM USING ARTIFICIAL INTELLIGENCE, METHOD OF CONTROLLING THE SAME, AND RECORDING MEDIUM

(71) Applicant: BENILIGHT CO., LTD., Cheonan-si (KR)

(72) Inventor: Yo Sik SONG, Cheonan-si Chungcheongnam-do (KR)

(21) Appl. No.: 18/894,694

(22) Filed: Sep. 24, 2024

Related U.S. Application Data
(63) Continuation of application No. PCT/KR2023/017441, filed on Nov. 3, 2023.

(30) Foreign Application Priority Data
Sep. 6, 2023 (KR) 10-2023-0118253

(51) Int. Cl. A61N 5/06; G16H 10/20; G16H 20/40

(52) U.S. Cl. CPC A61N 5/06; G16H 10/20; G16H 20/40; A61N 2005/0626; A61N 2005/0651

(57) ABSTRACT

Described are a light emitting diode (LED) phototherapy device control system using artificial intelligence (AI), a method of controlling the same, and a recording medium, the LED phototherapy device control system including: an LED pad to which an LED module including a plurality of LEDs arranged in a certain pattern is mounted; an LED control module; and a user equipment configured to provide a driving parameter value for the LED module to the LED control module while interworking with the LED control module, wherein the user application calculates the driving parameter value based on the data table and information input through the UI, and provides the driving parameter value to the LED control module, and the LED control module receives the driving parameter value from the user equipment, and controls the operation of the LED module based on the received driving parameter value.

### CROSS-REFERENCE TO RELATED APPLICATION

This application is based on and claims priority under 35 U.S.C. § 119 to Korean Patent Application No. 10-2023-0118253 filed on Sep. 6, 2023, in the Korean Intellectual Property Office, the disclosure of which is incorporated by reference herein in its entirety.

### BACKGROUND

### Field

The disclosure relates to a light emitting diode (LED) phototherapy device control system using artificial intelligence (AI), a method of controlling the same, and a recording medium, and more particularly to technology for controlling an LED phototherapy device using the AI.

### Description of the Related Art

Although clinical research results have shown that conventional light emitting diode (LED) phototherapy devices are capable of treating very many diseases, the conventional LED phototherapy devices actually have only limited therapeutic effects on a few limited diseases. To use the conventional LED phototherapy device in the treatment, a user needs to input a code provided by a manufacturer or the like or select a desired mode.

### SUMMARY

To solve the foregoing problems, an aspect of the disclosure is to provide a light emitting diode (LED) phototherapy device control system capable of applying research results from clinical paper data on LED phototherapy effects to an LED phototherapy device through artificial intelligence (AI) technology, a method of controlling the same, and a recording medium.

According to an embodiment of the disclosure, a light emitting diode (LED) phototherapy device control system using artificial intelligence (AI) includes: an LED pad to which an LED module including a plurality of LEDs arranged in a certain pattern is mounted; an LED control module configured to control operations of the LED module; and a user equipment configured to provide a driving parameter value for the LED module to the LED control module while interworking with the LED control module, wherein the user equipment includes a memory with a data table in which clinical information is stored, a processor, and a user application stored in the memory and executed by the processor, the user application generates a user interface (UI) for a questionnaire, displays the UI on a display, calculates the driving parameter value based on the data table and information input through the UI, and provides the driving parameter value to the LED control module, and the LED control module receives the driving parameter value from the user equipment, and controls the operation of the LED module based on the received driving parameter value.

The clinical information of the data table may include at least four first clinical items among a disease, a body part, a symptom level, an age, a gender, the number of clinical patients, and a therapeutic effect; and at least four second clinical items among a wavelength, a frequency, peak power, a duty ratio, an operation time, and a therapy interval related to the phototherapy, and the clinical information may be extracted from a lot of clinical paper data.

The UI may include items such as a user's age, gender, diseases, body part, and symptom level, and the user application may identify a clinical case, of which data of the first clinical item matches the information input through the UI, in the data table, and calculate the driving parameter value based on the data of the second clinical item of the identified clinical case.

The user application may calculate the driving parameter value by weighting the data of the second clinical item of the clinical cases, of which the data of the first clinical item matches the information input through the UI, based on at least one between a value of the number of clinical patients and a value of the therapeutic effect.

Meanwhile, according to an embodiment of the disclosure, a method of controlling a light emitting diode (LED) phototherapy device using artificial intelligence (AI) includes: collecting clinical paper data on LED phototherapy; extracting data about at least four first clinical items among a disease, a body part, a symptom level, an age, a gender, the number of clinical patients, and a therapeutic effect, and at least four second clinical items among a wavelength, a frequency, peak power, a duty ratio, an operation time, and a therapy interval related to the phototherapy, from the collected clinical paper data; storing the extracted data about the first clinical item and the second clinical item as a data table in a memory; generating a first user interface (UI) for a questionnaire and displaying the first UI on a display; calculating a driving parameter value for an LED module based on the data table and information input through the first UI; and providing the calculated driving parameter value to the LED control module.

The first UI may include items such as an age, a gender, a disease, a body part, and a symptom level, and the calculation of the driving parameter value may include identifying a clinical case, of which data of the first clinical item matches the information input through the first UI, in the data table, and calculating the driving parameter value by weighting the data of the second clinical item of the identified clinical case based on at least one between a value of the number of clinical patients and a value of the therapeutic effect.

The method may further include: displaying a second UI regarding therapy satisfaction and displaying the second UI on the display; storing data input through the second UI; and generating and displaying a graph of changes over time in data regarding the symptom level input through the first UI and data regarding the therapy satisfaction.

### BRIEF DESCRIPTION OF THE DRAWINGS

FIG. 1 is a schematic diagram illustrating an environment for providing a light emitting diode (LED) phototherapy device control service using artificial intelligence (AI) according to an embodiment of the disclosure.

FIG. 2 is a schematic diagram of an LED phototherapy device control system using AI according to an embodiment of the disclosure

FIG. 3 is a schematic diagram of a user equipment 300 according to an embodiment of the disclosure.

FIG. 4 is a flowchart of a method of controlling an LED phototherapy device 400 using AI according to an embodiment of the disclosure.

FIG. 5 is an exemplary graph showing symptoms and satisfaction displayed by a user application for controlling an LED phototherapy device using AI according to an embodiment of the disclosure.

### DETAILED DESCRIPTION OF EMBODIMENTS

Below, specific embodiments of the disclosure will be described in detail with reference to the accompanying drawings.

Hereinafter, exemplary embodiments of the disclosure will be described in detail with reference to the accompanying drawings. However, detailed descriptions about known functions or configurations, which may obscure the gist of the disclosure, are omitted from the following descriptions and the accompanying drawings. Further, it is noted that like numerals refer to like elements throughout the accompanying drawings.

It should be understood that the terms used in this specification and the appended claims should not be construed as limited to typical and lexical meanings, but interpreted based on the meanings and concepts corresponding to technical aspects of the disclosure on the basis of the principle that the inventor is allowed to define terms appropriately for the best description. Therefore, the description proposed herein is merely a preferable example for the illustrative purpose only, not intended to limit the scope of the disclosure, so it should be understood that other equivalents and modifications could be made thereto without departing from the spirit and scope of the disclosure.

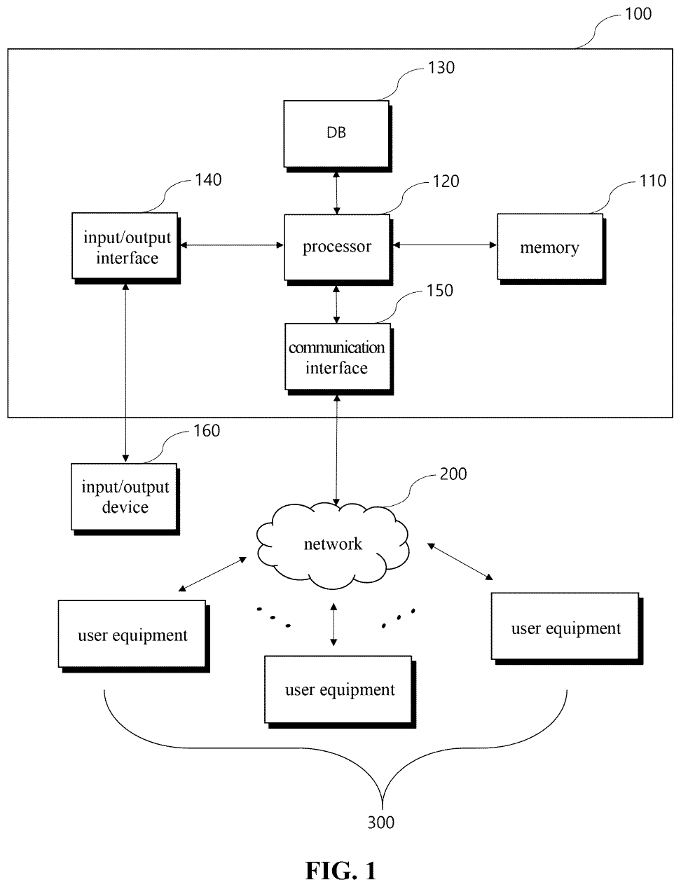

FIG. 1 is a schematic diagram illustrating an environment for providing a light emitting diode (LED) phototherapy device control service using artificial intelligence (AI) according to an embodiment of the disclosure. Referring to FIG. 1, the environment for providing the LED phototherapy device control service using the AI according to an embodiment of the disclosure includes a server 100, and a user equipment 300 interworking with the server 100 through a network 200.

The server 100 provides the LED phototherapy device control service using the AI according to an embodiment of the disclosure, and exchanges data with the Internet or the user equipment 300 with an application installed therein, thereby providing the LED phototherapy device control service. The server 100 includes a memory 110 in which data and computer program codes are stored, and a processor 120 by which the computer program codes are executed. The server 100 includes a database (DB) 130 in which clinical information and member information are stored according to diseases. The server 100 collects clinical paper data on phototherapy online, analyzes the clinical paper data to extract the clinical information, and constructs a data table. Clinical information constructed as the data table includes at least four first clinical items among a disease, a body part, a symptom level, an age, a gender, the number of clinical patients, and a therapeutic effect; and at least four second clinical items among a wavelength, a frequency, peak power, a duty ratio, an operation time, and a therapy interval related to the phototherapy. The first clinical item refers to information related to a patient, and the second clinical item refers to information about optical parameter values related to the phototherapy. The data collection and extraction from such clinical paper data may be performed periodically or at any time by an AI analysis module. Additionally, at least part of the constructed data table may be transmitted to the user terminal 300 and updated periodically or at any time as necessary.

The server 100 includes a parameter calculation model to calculate a driving parameter value of an LED module according to patients, and the parameter calculation model calculates the driving parameter value for the LED module according to the logic set based on the clinical information constructed as the data table. The parameter calculation model may be implemented as an algorithm and may be updated periodically or at any time as necessary. According to an embodiment, at least part of the parameter calculation model may be transmitted to and installed on the user equipment 300 and function in the user equipment 300. According to another embodiment, the parameter values calculated by the server may be transmitted to the user equipment 300.

Meanwhile, the server 100 may manage the member information, a therapy history, etc. by interworking with the user equipment 300 and provide customized the therapy information to the patients.

The server 100 exchanges data with the user equipment 300 through the network 200, such as the Internet, and includes a communication module for network communication. Further, the server may include an input/output device 160 for a user's input and output, for example, a microphone, a keyboard, a mouse, a display, etc. and an input/output interface 140 for interfacing with the input/output device 160. A communication interface 150 may for example include the communication module for one or more networks among a personal area network (PAN), a local area network (LAN), a campus area network (CAN), a metropolitan area network (MAN), a wide area network (WAN), a broadband network (BBN), the Internet, etc.

The user equipment 300 may include a mobile terminal such as a smartphone or a tablet PC, a PC, etc., and have a user application installed to receive services according to embodiments of the disclosure. For example, the user equipment 300 may receive the services provided by the server 100 by interworking with the server 100 under the control of the application. The user application installed on the user equipment 300 may be divided into an app for the smartphone and a web application for the PC.

Meanwhile, a file distribution server may be provided to distribute a file for the installation of the user application. For example, the file distribution server may store and manage the file, and provide the stored file to the user equipment 300 in response to a request from the user equipment 300, and the user equipment 300 may install the application based on the file provided by the file distribution server and receive the service through the installed user application. The file distribution server may be a server involved in the server 100, but may also be a separate server linked to the server 100 as a server of a third party.

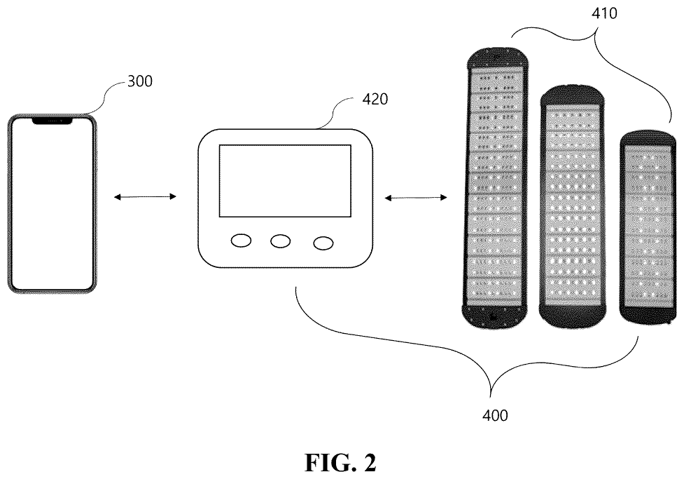

FIG. 2 is a schematic diagram of an LED phototherapy device control system using AI according to an embodiment of the disclosure. Referring to FIG. 2, the LED phototherapy device control system using the artificial intelligence according to an embodiment of the disclosure includes the user equipment 300, and the LED phototherapy device 400.

The LED phototherapy device 400 includes a LED pad 410, and an LED control module 420. The LED pad 410 is configured in such a way that an LED module including a plurality of LEDs arranged in a certain pattern on a printed circuit board is mounted to a pad.

The LED control module 420 is to control the operation of the LED module, and includes a micro controller such as an MCU. The LED control module 420 may be connected to the LED module by a wire or wirelessly. Meanwhile, the LED control module 420 may include a user interface (UI) means such as a button or a touch panel display. A user may get the phototherapy of the LED pad 410 by selecting a desired menu or mode using the button or touch panel provided on the LED control module 420.

The LED control module (420) includes a wireless communication module, such as a Bluetooth communication module, for communication with a user equipment (300).

The user equipment 300 is to provide the driving parameter value, i.e., a control value for the LED module to the LED control module 420 while interworking with the LED control module 420, and has the user application installed thereon to control the LED phototherapy device according to the disclosure. The user equipment 300 and the LED control module 420 may communicate with each other through a wireless communication module such as Bluetooth communication module.

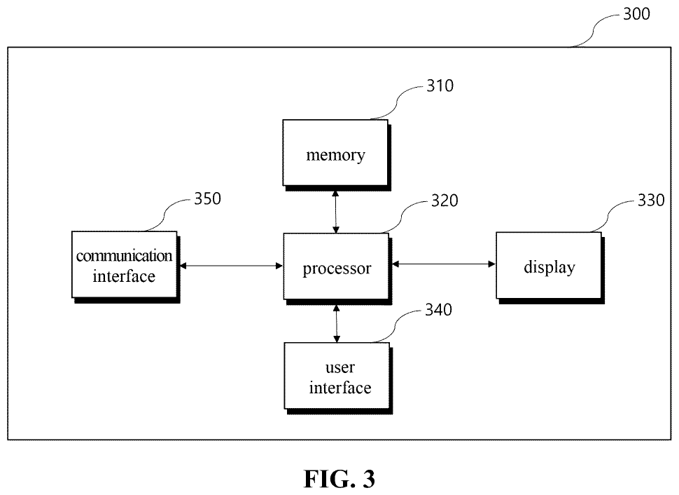

FIG. 3 is a schematic diagram of a user equipment 300 according to an embodiment of the disclosure. Referring to FIG. 3, the user equipment 300 includes a memory 310 in which data and program codes are stored, and a processor 320 by which the program codes are executed.

The memory 310 refers to a computing device-readable recording medium, and may store data and at least one computer program code to be executed by the processor 320. The computer program code may be loaded from a floppy drive, a disk, a tape, a DVD/CD-ROM drive, a memory card, etc. separate from the memory 310 into the memory 310

The processor 320 is to execute and process computer program instructions by performing basic logic, calculations, operations, etc., and the computer program code stored in the memory 310 is loaded into and executed by the processor 320. The processor 320 executes the user application stored in the memory 310 and performs a series of functions to control the LED phototherapy device.

The memory 310 includes the data table in which the clinical information according to the diseases is tabulated. For example, the data table may be constructed in the server 100 and at least part thereof may be provided to the user equipment 300. As another example, the data table of the clinical information described above may be constructed in the user equipment 300. A series of processes such as collecting and extracting the clinical paper data online to construct the data table may be performed by the user application. The user application includes an AI analysis module to process a series of processes such as collecting and extracting the clinical paper data, and detailed descriptions thereof will be replaced with the foregoing description about the server 100 to avoid redundant description.

The memory 310 includes the parameter calculation model to calculate the driving parameter value for the LED module according to patients. The parameter calculation model is provided by the server 100 and stored in the memory 310, and the processor 320 uses the parameter calculation model to calculate the driving parameter values for the LED module according to the preset logic based on the clinical information constructed as the data table. The parameter calculation model stored in the user equipment 300 may be updated by the server 100 periodically or at any time as necessary.

As shown in FIG. 3, the user equipment 300 according to an embodiment of the disclosure may further include a display 330 for displaying data and images, a user interface 340 for receiving a user's input, and a communication interface 350 for interworking with the outside.

Below, a method of controlling the LED phototherapy device using the AI according to an embodiment of the disclosure will be described. Repetitive descriptions to the foregoing embodiment will be avoided as necessary.

First, a user accesses the file distribution server or the server 100 through the user equipment 300, downloads the user application according to the disclosure, and installs and executes the user application. Then, a user may sign up as a member, and register information such as a gender, an age, diseases, and symptom levels when signing up. In this case, multiple users may be registered in the application installed on one user equipment 300. The user application installed on the user equipment 300 may contain the parameter calculation model for the LED phototherapy device 400 and be updated by the server 100 periodically or at any time.

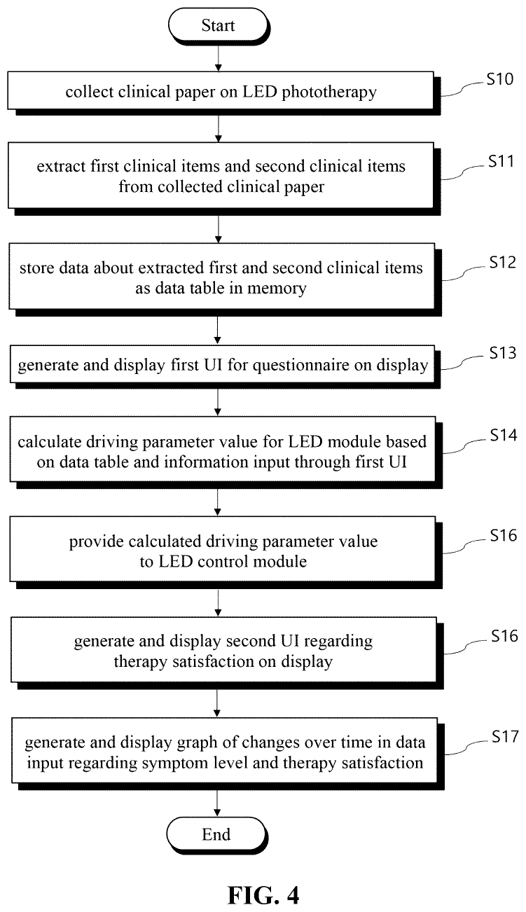

FIG. 4 is a flowchart of the method of controlling the LED phototherapy device 400 using the AI according to an embodiment of the disclosure.

The user equipment 300 (or user application) collects the clinical paper data on the LED phototherapy online (S10). The user application may be set to periodically search specific sites and extract relevant clinical paper data.

The user equipment 300 (or the user application) extracts at least four first clinical items among a disease, a body part, a symptom level, an age, a gender, the number of clinical patients, and a therapeutic effect; and at least four second clinical items among a wavelength, a frequency, peak power, a duty ratio, an operation time, and a therapy interval related to the phototherapy from the collected clinical paper data (S11).

As described above, the first clinical item refers to information related to a patient, and the second clinical item refers to information about optical parameter values related to the phototherapy. The user application includes the AI analysis module for collecting and extracting the data from such clinical paper data, and may be set to perform operations periodically or at any time.

The user equipment 300 (or the user application) constructs the data about the extracted first and second clinical items as the data table and stores the data table in the memory 310 (S12).

The table 1 below shows an example of the data table about the clinical information according to an embodiment of the disclosure.

TABLE 1

| | | | Symptom | | Gender | | | | | | | | Therapeutic | Note |
|---|---|---|---|---|---|---|---|---|---|---|---|---|---|---|
| | | Body | level | | (male, | clinical | | LED | Duty | | Operation | | effect eₙ | (LED/ |
| | Diseases | part | (1to10) | Age | female) | patients | Wavelength | power | ratio | Freq. | time | Interval | (1to5) | laser) |
| 1 | Alzheimer 1 | head | 4 | 50 | F | 12 | W₁ | P₁ | D₁ | F₁ | T₁ | Once | 3 | . . . |
| | | | | | | | | | | | | a day | | |
| 2 | Alzheimer 2 | | 6 | 40 | M | 50 | W₂ | P₂ | D₂ | F₂ | T₂ | Once | 4 | . . . |
| | | | | | | | | | | | | every | | |
| | | | | | | | | | | | | 2 days | | |
| 3 | Alzheimer 3 | | 8 | 60 | F | 3 | W₃ | P₃ | D₃ | F₃ | T₃ | Once | 5 | . . . |
| | | | | | | | | | | | | every | | |
| | | | | | | | | | | | | 3 days | | |
| 4 | Cognitive | | . . . | . . . | . . . | . . . | . . . | . . . | . . . | . . . | . . . | . . . | . . . | . . . |
| | impairment 1 | | | | | | | | | | | | | |
| 5 | Cognitive | | . . . | . . . | . . . | . . . | . . . | . . . | . . . | . . . | . . . | . . . | . . . | . . . |
| | impairment 2 | | | | | | | | | | | | | |
| 6 | . . . | . . . | . . . | . . . | . . . | . . . | . . . | . . . | . . . | . . . | . . . | . . . | . . . | . . . |
| . . . | . . . | . . . | . . . | . . . | . . . | . . . | . . . | . . . | . . . | . . . | . . . | . . . | . . . | . . . |
| N | . . . | . . . | . . . | . . . | . . . | . . . | . . . | . . . | . . . | . . . | . . . | . . . | . . . | . . . |

As shown in the Table 1 above, the clinical data corresponding to each clinical item is extracted from the clinical paper data and provided as the data table. The foregoing embodiment describes the operations S10 to S12 are performed by the user equipment 300. However, according to an alternative embodiment of the disclosure, the operations S10 to S12 may also be performed by the server 100. In the latter case, the server 100 provides the data table to the user equipment 300, and the user equipment 300 stores the data table in the memory 310 and uses the data table to calculate the driving parameter value later.

When a user wants to get the therapy using the LED phototherapy device 400 and logs in by executing the user application installed on the user equipment 300, the user application generates a first UI for conducting a questionnaire to the user and display the first UI on the display (S13).

The first UI includes items about the user's age (group), gender, diseases, body part, and symptom level. When a user has input information for all or part of those items upon signing up as a member, the questionnaire regarding the previously input information may be omitted. Further, for example, the symptom level is varied depending on the therapy, and therefore the user application displays the previously input information on the first UI when a user uses the LED phototherapy device 400, thereby allowing the user to edit only the items that the user wishes to modify.

The first UI may further include an item asking whether a user has a condition such as cancer or pregnancy. If ‘yes’ is selected in this item, the use of the LED phototherapy device 400 may be restricted. As another example, if a user has multiple diseases, the user may input and register the information for each disease.

The user application calculates the driving parameter value for the LED module based on the data table and the information input through the first UI (S14). Here, the driving parameters for the LED module include at least four among the wavelength (or the frequency), the peak power, the duty ratio, the operation time, and the therapy interval for the LED module.

It will be described later how the user application calculates the driving parameter value for the LED module. In general, the driving parameter value for the LED module used in the phototherapy devices is limited to a certain range. In this embodiment, the wavelength of the LED module: 660 nm, 850 nm, 940 nm, LED emission power: Pₘᵢₙ to Pₘₐₓ (0 to 15 mW), the duty ratio: 0 to 100%, driving frequency: Fₘᵢₙ to Fₘₐₓ (0 to 5 kHz), operation time: Tₘᵢₙ to Tₘₐₓ (0 to 15 minutes) are set by way of example.

The user application identifies a clinical case, of which the data of the first clinical item matches the information input through the first UI, in the data table. Then, the driving parameter value for the LED module is calculated based on the data of the second clinical item of the identified clinical case.

For example, when a user inputs ‘Alzheimer’ for the disease, ‘head’ for the body part, ‘60s’ for the age, ‘female’ for the gender, and ‘6’ for the symptom level in Table 1, the user application extracts the clinical cases whose values match those of the corresponding item from the data table. For example, when there are three matching clinical cases, the user application calculates the driving parameter value for the LED module based on the data of the second clinical items (the LED driving parameters) of the three clinical cases.

Specifically, the driving parameter value is calculated by weighting the data of the second clinical item of the clinical cases, of which the data of the first clinical item matches the information input through the first UI, according to at least one between the value of the number of clinical patients and the value of the therapeutic effect. For example, a final LED driving parameter value is calculated by assigning higher weight to the second clinical item (LED driving parameter) of the clinical case with a large number of clinical patients and/or the clinical case with a high therapeutic effect.

According to the disclosure, for example, the peak power (P) for the LED module may be calculated by the following Equation.

$$P = ( {P}_{1} {e}_{1} + {P}_{2} {e}_{2} + {P}_{3} {e}_{3} ) / ( {e}_{1} + {e}_{2} + {e}_{3} ) \qquad [ Equation 1 ]$$

where, P is a peak power value of the clinical case that matches the information input by a user answering the questionnaire in the data table, and e is a weight value ranging from 1 to 5 given based on the number of clinical patients and the therapeutic effect of the matched clinical case (the minimum value is 1). For example, the larger the number of clinical patients, the higher the weighted value, and the higher the therapeutic effect, the higher the weighted value.

The user application identifies whether the calculated P value is out of a specification range of the LED phototherapy device 400 as shown below, and then identifies the final power peak value.

For example, (i) if Pₘᵢₙ≤P≤Pₘₐₓ, the calculated P is identified as the final power peak value, (ii) if P<Pₘᵢₙ, Pₘᵢₙ is identified as the final power peak value, and (iii) if Pₘₐₓ<P, Pₘₐₓ is identified as the final power peak value.

According to the disclosure, for example, the duty ratio (D) for the LED module may be calculated using the following Equation.

$$D = ( {D}_{1} {e}_{1} + {D}_{2} {e}_{2} + {D}_{3} {e}_{3} ) / ( {e}_{1} + {e}_{2} + {e}_{3} ) \qquad [ Equation 2 ]$$

where, D is a duty ratio value of the clinical case that matches the information input by a user answering the questionnaire in the data table, and e is a weight value ranging from 1 to 5 given based on the number of clinical patients and the therapeutic effect of the matched clinical case in the data table. For example, the larger the number of clinical patients, the higher the weighted value, and the higher the therapeutic effect, the higher the weighted value.

According to the disclosure, for example, the driving frequency (F) for the LED module may be calculated using the following Equation.

$$F = ( {F}_{1} {e}_{1} + {F}_{2} {e}_{2} + {F}_{3} {e}_{3} ) / ( {e}_{1} + {e}_{2} + {e}_{3} ) \qquad [ Equation 3 ]$$

where, F is a frequency value of the clinical case that matches the information input by a user answering the questionnaire in the data table, and e is a weight value ranging from 1 to 5 given based on the number of clinical patients and the therapeutic effect of the matched clinical case in the data table. For example, the larger the number of clinical patients, the higher the weighted value, and the higher the therapeutic effect, the higher the weighted value.

The user application identifies whether the calculated F value is out of a specification range of the LED phototherapy device 400 as shown below, and then identifies the final driving frequency value.

For example, (i) if Fₘᵢₙ≤F & Fₘₐₓ, the calculated F is identified as the final driving frequency value, (ii) if F<Fₘᵢₙ, Fₘᵢₙ is identified as the final driving frequency value, and (iii) if Fₘₐₓ<F, Fₘₐₓ is identified as the final driving frequency value.

According to the disclosure, for example, the operation time (T) for the LED module may be calculated using the following Equation.

$$T = ( {T}_{1} {e}_{1} + {T}_{2} {e}_{2} + {T}_{3} {e}_{3} ) / ( {e}_{1} + {e}_{2} + {e}_{3} ) \qquad [ Equation 4 ]$$

where, T is an operation time value of the clinical case that matches the information input by a user answering the questionnaire in the data table, and e is a weight value ranging from 1 to 5 given based on the number of clinical patients and the therapeutic effect of the matched clinical case in the data table. For example, the larger the number of clinical patients, the higher the weighted value, and the higher the therapeutic effect, the higher the weighted value.

The user application identifies whether the calculated T value is out of a specification range of the LED phototherapy device 400 as shown below, and then identifies the final operating time value.

For example, (i) if Tₘᵢₙ≤T≤Tₘₐₓ, the calculated T is identified as the final operation time value, (ii) if T<Tₘᵢₙ, Tₘᵢₙ is identified as the final operation time value, and (iii) if Tₘₐₓ<T, Tₘₐₓ is identified as the final operation time value.

The user equipment 300 (or the user application) transmits the parameter values calculated through the foregoing processes, for example, the wavelength, the frequency, the peak power, the duty ratio, the operation time, and the therapy interval for the LED module to the LED control module 420 (S15). The LED control module 420 controls the operations of the LED module based on the LED driving parameter values received through the user equipment 300.

In this way, according to the disclosure, the operation of the LED phototherapy device 400 is controlled by the driving parameter values corresponding to the optimal therapeutic effect based on the clinical information that matches the information such as the diseases, the body part, the age, the gender, and the symptom level input by a user so that the user can get the therapy under optimal conditions.

When the therapy is completed, the user application generates a second UI regarding the therapy satisfaction and displays the second UI on the display (S16). Then, changes over time in data input by a user regarding the symptom level and the therapy satisfaction are generated and displayed as a graph (S17).

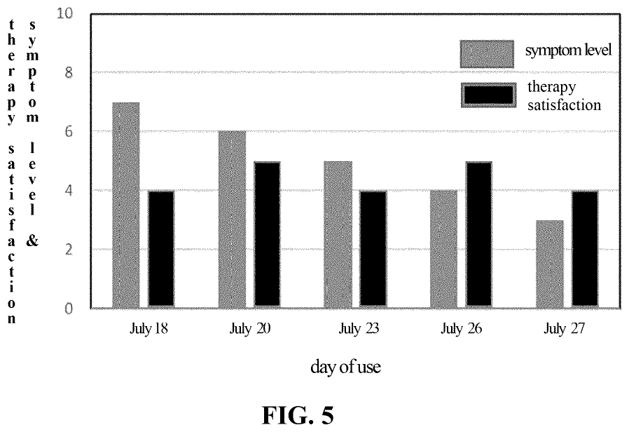

FIG. 5 is an exemplary graph showing symptoms and satisfaction displayed by the user application for controlling the LED phototherapy device 400 using AI according to an embodiment of the disclosure. As shown in FIG. 5, by displaying the therapy progress over time at a glance, a user can check how symptoms or satisfaction have changed during his/her therapy period.

The disclosure may be implemented by various computer-readable recording media, such as a magnetic storage medium, an optical readout medium, and a digital storage medium, in which a computer program for performing the method of controlling the LED phototherapy device 400 according to the disclosure is stored, when executed on a computer. Further, the subject matters expressed as steps in the appended claims are not limited to that order of steps.

As described above, the LED phototherapy device control system using the AI according to the disclosure, the method of controlling the same, and the recording medium allow a user to easily treat various diseases at home using a phototherapy control system to which the AI technology is applied.

Although a few embodiments of the disclosure have been described so far, a person having ordinary knowledge in the art can understand that those embodiments are partially modified or replaced without departing from the technical spirit of the disclosure. Therefore, the scope of the disclosure should be regarded as affecting the subject matters described in the appended claims and its equivalents.

What is claimed is:

1. A light emitting diode (LED) phototherapy device control system using artificial intelligence (AI), the LED phototherapy device control system comprising:
an LED pad to which an LED module comprising a plurality of LEDs arranged in a certain pattern is mounted;
an LED control module configured to control operations of the LED module; and
a user equipment configured to provide a driving parameter value for the LED module to the LED control module while interworking with the LED control module, wherein
the user equipment comprises a memory with a data table in which clinical information is stored, a processor, and a user application stored in the memory and executed by the processor,
the user application generates a user interface (UI) for a questionnaire, displays the UI on a display, calculates the driving parameter value based on the data table and information input through the UI, and provides the driving parameter value to the LED control module, and
the LED control module receives the driving parameter value from the user equipment, and controls the operation of the LED module based on the received driving parameter value.

2. The LED phototherapy device control system of claim 1, wherein
the clinical information of the data table comprises at least four first clinical items among a disease, a body part, a symptom level, an age, a gender, the number of clinical patients, and a therapeutic effect; and at least four second clinical items among a wavelength, a frequency, peak power, a duty ratio, an operation time, and a therapy interval related to the phototherapy, and
the clinical information is extracted from a lot of clinical paper data.

3. The LED phototherapy device control system of claim 2, wherein
the UI comprises items such as a user's age, gender, diseases, body part, and symptom level, and
the user application identifies a clinical case, of which data of the first clinical item matches the information input through the UI, in the data table, and calculates the driving parameter value based on the data of the second clinical item of the identified clinical case.

4. The LED phototherapy device control system of claim 3, wherein the user application calculates the driving parameter value by weighting the data of the second clinical item of the clinical cases, of which the data of the first clinical item matches the information input through the UI, based on at least one between a value of the number of clinical patients and a value of the therapeutic effect.

5. A method of controlling a light emitting diode (LED) phototherapy device using artificial intelligence (AI), the method comprising:
collecting clinical paper data on LED phototherapy;
extracting data about at least four first clinical items among a disease, a body part, a symptom level, an age, a gender, the number of clinical patients, and a therapeutic effect, and at least four second clinical items among a wavelength, a frequency, peak power, a duty ratio, an operation time, and a therapy interval related to the phototherapy, from the collected clinical paper data;
storing the extracted data about the first clinical item and the second clinical item as a data table in a memory;
generating a first user interface (UI) for a questionnaire and displaying the first UI on a display;
calculating a driving parameter value for an LED module based on the data table and information input through the first UI; and
providing the calculated driving parameter value to the LED control module.

6. The method of claim 5, wherein
the first UI comprises items such as an age, a gender, a disease, a body part, and a symptom level, and
the calculation of the driving parameter value comprises identifying a clinical case, of which data of the first clinical item matches the information input through the first UI, in the data table, and calculating the driving parameter value by weighting the data of the second clinical item of the identified clinical case based on at least one between a value of the number of clinical patients and a value of the therapeutic effect.

7. The method of claim 6, further comprising:
displaying a second UI regarding therapy satisfaction and displaying the second UI on the display;
storing data input through the second UI; and
generating and displaying a graph of changes over time in data regarding the symptom level input through the first UI and data regarding the therapy satisfaction.

8. A computer-readable recording medium in which a program for performing a method of controlling a light emitting diode (LED) phototherapy device using artificial intelligence (AI) according to claim 5.

9. A computer-readable recording medium in which a program for performing a method of controlling a light emitting diode (LED) phototherapy device using artificial intelligence (AI) according to claim 6.

10. A computer-readable recording medium in which a program for performing a method of controlling a light emitting diode (LED) phototherapy device using artificial intelligence (AI) according to claim 7.
